(19) United States
(12) Patent Application Publication
(10) Pub. No.: US 2024/0319194 A1
(43) Pub. Date: Sep. 26, 2024

(54) DIAGNOSIS AND TREATMENT OF PANCREATIC CANCER

(71) Applicant: Sheba Impact Ltd., Ramat-Gan (IL)

(72) Inventors: Doron YABLECOVITCH, Tel Aviv (IL); Ido LAISH, Kiryat-Ono (IL)

(73) Assignee: Sheba Impact Ltd., Ramat-Gan (IL)

(21) Appl. No.: 18/574,790

(22) PCT Filed: Jun. 29, 2022
(86) PCT No.: PCT/IL2022/050700

§ 371 Date: Dec. 28, 2023

Related U.S. Application Data
(60) Provisional application No. 63/216,071, filed on Jun. 29, 2021.

(51) Int. Cl. G01N 33/574; A61K 45/06

(52) U.S. Cl. CPC G01N 33/57438; A61K 45/06

(57) ABSTRACT

A method of diagnosing pancreatic cancer in a subject in need thereof is provided. The method comprising determining a level of at least syndecan 1 (SDC1) in a blood sample or a fraction thereof of the subject, wherein when the level of SDC1 is above a predetermined threshold, the subject is diagnosed with pancreatic cancer.

### RELATED APPLICATION/S

This application claims priority from U.S. Provisional Patent Application No. 63/216,071 filed Jun. 29, 2021, which is hereby incorporated by reference in its entirety.

### FIELD AND BACKGROUND OF THE INVENTION

The present invention, in some embodiments thereof, relates to diagnosis and treatment of pancreatic cancer.

Pancreatic ductal adenocarcinoma (PDAC) is a leading cause of cancer death with dismal prognosis (1). Despite improvement in therapeutic strategies, the estimated five-year survival rate is only 8%, mainly because it is usually diagnosed at very late stages (2). This is explained by its asymptomatic nature at early stages, together with the lack of efficient screening tests for early detection (3, 4). The earliest genetic event in the progression of the normal ductal epithelia to premalignant pancreatic intraepithelial neoplasia (PanIN) is the mutation of the KRAS oncogene, which functions as a driver in the tumorigenesis (5-7). Currently, the only broadly used biomarker for PDAC, CA 19-9 has multiple limitations and there is an unmet need for novel biomarkers (8, 9). Liquid biopsies, including circulating tumor cells, cell-free DNA or methylated DNA, had been suggested to be used as early-stage diagnostics, as well as prognostic biomarkers, but their low-level at early stage PDAC have presented a barrier to use in diagnosis (10). Analyzing the metabolome is another novel approach for the detection of cancer signatures, as patients may have different metabolic profile, but there is a need for further standardizations and validation (11-13).

Syndecan-1 (SDC1), a member of the transmembrane heparan sulfate proteoglycans (HSPGs) family, is predominantly expressed on the basolateral membrane surface of epithelial cells (14). It mediates cell adhesion, participates in cell proliferation, migration and cell-matrix interaction, and promoting wound healing by regulation of immune function (15, 16). During infection, inflammation, and tissue injury, serum levels of SDC1 increase sharply, contributing to diverse pathophysiological events. (17-21).

In the context of tumorigenesis, it was shown that SDC1 can regulate multiple functions, including tumor cell attachment, growth, proliferation, and angiogenesis through different signaling pathways (e.g, activation of Wnt pathway) (22-23). Altered SDC1 expression has thus been associated with the presence and progression of various tumors (24-27), specifically in PDAC, as was recently published in a landmark study by Yao and colleagues. SDC1 is upregulated at the cell surface by oncogenic KRAS, where it regulates macropinocytosis, a critical metabolic pathway that fuels PDAC cell growth and promotes tumor progression (28). In another study, patients carrying KRAS somatic mutations had a higher SDC1 mRNA expression than those without mutations, suggesting a role for SDC1 as a KRAS effector and expression signature (29). SDC1 expression in situ was suggested as a novel prognostic marker in pancreatic cancer. Oncology. 2005; 68:97-106.

Hence, the use of SDC1 as an in-situ marker has been proposed by way of its expression on tumor cells. But its expression varies in other tissues even if closely related to the tumor, such as in stromal cells (Akl et al. Oncotarget. 2015; 6:28693-28715). Interestingly, Akl et al. report that epithelial SDC1 has been observed in most human pancreatic carcinoma samples date, and expression is predictive of a more favorable prognosis in patients undergoing curative surgery. However, stromal SDC1 expression was weak or negative in over 60% of the tumors evaluated, and lack of stromal expression predicted a better prognosis in these patients.

### SUMMARY OF THE INVENTION

According to an aspect of some embodiments of the present invention there is provided a method of diagnosing pancreatic cancer in a subject in need thereof, the method comprising determining a level of syndecan 1 (SDC1) in a blood sample or a fraction thereof of the subject, wherein when the level of SDC1 is above a predetermined threshold, the subject is diagnosed with pancreatic cancer.

According to an aspect of some embodiments of the present invention there is provided a method of prognosing survival of a subject diagnosed with pancreatic cancer, the method comprising determining a level of syndecan 1 (SDC1) in a blood sample or a fraction thereof of the subject, wherein when the level of SDC1 is above a predetermined threshold, poor survival is indicated.

According to an aspect of some embodiments of the present invention there is provided a method of treating pancreatic cancer in a subject in need thereof, the method comprising:
(a) diagnosing pancreatic cancer in the subject as described herein; and
(b) treating the subject with an anti pancreatic cancer therapy or selecting a treatment with an anti pancreatic cancer therapy.

According to an aspect of some embodiments of the present invention there is provided a method of monitoring treatment of pancreatic cancer in a subject in need thereof, the method comprising:
(a) treating the subject with anti pancreatic cancer therapy;
(b) determining a level of syndecan 1 (SDC1) in a blood sample or a fraction thereof of the subject prior to and following (a), wherein when a decrease in SDC1 is determined an efficacious treatment is indicated.

According to an aspect of some embodiments of the present invention there is provided a composition of matter comprising a blood sample or a fraction thereof of a subject and an antibody to SDC1.

According to some embodiments of the invention, the blood sample of fraction thereof is of a subject having pancreatic cancer.

According to some embodiments of the invention, the pancreatic cancer is pancreatic ductal adenocarcinoma (PDAC).

According to some embodiments of the invention, the level is protein level.

According to some embodiments of the invention, the determining is by ELISA.

According to some embodiments of the invention, the pancreatic cancer is KRAS+.

According to some embodiments of the invention, the pancreatic cancer is resectable.

According to some embodiments of the invention, the treating is by a surgery.

According to some embodiments of the invention, a location of the cancer is in the head of the pancreas.

According to some embodiments of the invention, the method further comprises corroboration of the pancreatic cancer or treatment thereof with imaging or molecular markers.

According to some embodiments of the invention, the molecular markers are selected from the group consisting of HPA, MMP and CA 19-9.

According to some embodiments of the invention, the subject does not suffer from an autoimmune disease, a systemic active infectious disease and/or an extra-pancreatic malignancy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a box-plot representation of median serum syndecan-1 levels in patients with pancreatic ductal adenocarcinoma (PDAC) versus healthy controls (derivation cohort).

FIG. 1B is a box-plot representation of median serum syndecan-1 levels in patients with pancreatic ductal adenocarcinoma (PDAC) versus healthy controls (validation cohort).

FIG. 2A is a receiver operating characteristic curve of the diagnostic accuracy of serum syndecan-1 to discriminate between healthy controls and patients with pancreatic ductal adenocarcinoma (derivation cohort).

FIG. 2B is a receiver operating characteristic curve of the diagnostic accuracy of serum syndecan-1 to discriminate between healthy controls and patients with pancreatic ductal adenocarcinoma (validation cohort).

FIG. 3A is a box-plot representation of median serum syndecan-1 levels at diagnosis in patients with pancreatic ductal adenocarcinoma according to tumor stages compared with healthy controls. Serum syndecan-1 levels were not significantly different among the different stage groups (but significant vs. control).

FIG. 3B is a box-plot representation of median serum syndecan-1 levels at diagnosis in patients with metastatic pancreatic ductal adenocarcinoma (PDAC) compared with non-metastatic PDAC patients and healthy controls.

FIG. 4 is a Kaplan-Meier curve of overall survival among patients with serum syndecan-1 levels of 35 ng/ml or more at baseline versus those with baseline serum syndecan-1 levels lower than 35 ng/ml. Log rank test for equality of survivor functions, p value=0.15.

### DESCRIPTION OF SPECIFIC EMBODIMENTS OF THE INVENTION

The present invention, in some embodiments thereof, relates to diagnosis and treatment of pancreatic cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Syndecan-1 (SDC1) is involved in the regulation of multiple functions in the tumorigenesis, specifically in pancreatic cancer. However in contrast to its in situ behavior as a marker for pancreatic cancer, no work has been reported to date with respect to serum SDC1 in pancreatic cancer and in fact its levels in neighboring tissues were found not to correlate with disease.

In order to evaluate the feasibility of using SDC1 as a serum marker for pancreatic cancer, patients with a new diagnosis of biopsy-proven PDAC were enrolled along-side healthy individuals, in a derivation-validation cohort design. Serum SDC1 baseline levels were quantified by ELISA. The diagnostic accuracy of SDC1 level for diagnosis of PDAC was computed. In a unified cohort, enriched with additional early-stage PDAC, a subsequent analysis evaluated the association of this biomarker with survival outcomes and with patient and tumor characteristics.

In the derivation cohort, serum SDC-1 level was significantly higher in PDAC patients (n=39) compared with healthy controls (n=20) (40.1 ng/ml, IQR 29.8-95.3 vs. 25.6 ng/ml, IQR 17.1-29.8, respectively; p<0.0001). The ROC analysis area under the curve (AUC) was 0.847 (95% CI 0.747 to 0.947, p<0.0001). These results were replicated in the validation cohort (n=38 PDAC, n=20 controls). In the combined-enriched PDAC cohort (n=110), using a cutoff of 35 ng/ml the median overall 5-year survival between patients below and above this cutoff, was not significantly different. Serum SDC-1 levels associated with tumor location at the head of pancreas vs. body/tail localization (p=0.019). SDC1 levels were elevated in 12/20 PDAC patients who had normal CA19-9 levels.

Thus is may be safely concluded that serum SDC1 is a promising biomarker for early blood-based diagnosis of pancreatic cancer, possibly facilitating earlier life-saving interventions.

Thus, according to an aspect of the invention there is provided a method of diagnosing pancreatic cancer in a subject in need thereof, the method comprising determining a level of syndecan 1 (SDC1) in a blood sample or a fraction thereof of the subject, wherein when said level of SDC1 is above a predetermined threshold, the subject is diagnosed with pancreatic cancer.

According to an additional or an alternative aspect there is provided a method of prognosing survival of a subject diagnosed with pancreatic cancer, the method comprising determining a level of syndecan 1 (SDC1) in a blood sample or a fraction thereof of the subject, wherein when said level of SDC1 is above a predetermined threshold, poor survival is indicated.

According to an additional or an alternative aspect there is provided a method of treating pancreatic cancer in a subject in need thereof, the method comprising:
(a) diagnosing pancreatic cancer in the subject as described herein; and
(b) treating the subject with an anti pancreatic cancer therapy or selecting a treatment with an anti pancreatic cancer therapy.

It will be appreciated that SDC1 can be determined (measured) alone or in combination with other markers, which will improve the diagnostic/prognostic values of the disease in an additive or synergic manner. For example, when combined with MMP7, results in the derivation cohort, show the AUC upon ROC analysis for is 0.925.

As used herein “pancreatic ductal adenocarcinoma” (PDAC) is a type of exocrine pancreatic cancer. It develops from cells lining small tubes in the pancreas called ducts (duct cells in the diagram above). These carry the digestive juices, which contain enzymes, into the main pancreatic duct and then on into the duodenum (first part of the small intestine). PDAC can grow anywhere in the pancreas, although it is most often found in the head of the pancreas.

Staging of PDAC can be done using any method which is acceptable by regulatory agencies. Thus for example, the cancer can be classified either by TMN system/American Joint Committee on Cancer (AJCC) Staging for PDAC, or by three clinically distinct patient groups—resectable (T1-3, stages 1 and 2), locally advanced (T4, stage 3) and metastatic disease (M1, stage 4). According to some embodiments, staging is determined on the basis of imaging modalities (typically CT scans) for non-resectable tumors, and pathologically-determined for resectable ones.

According to the American Cancer Society the TMN staging is as in Table A which follows.

TABLE A
Stages of pancreatic cancer

| AJCC | Stage | |
|---|---|---|
| Stage | grouping | Stage description* |
| 0 | Tis | The cancer is confined to the top layers of pancreatic duct cells and has not invaded |
| | N0 | deeper tissues. It has not spread outside of the pancreas. These tumors are sometimes |
| | M0 | referred to as carcinoma in situ (Tis). |
| | | It has not spread to nearby lymph nodes (N0) or to distant sites (M0). |
| IA | T1 | The cancer is confined to the pancreas and is no bigger than 2 cm (0.8 inch) across (T1). |
| | N0 | It has not spread to nearby lymph nodes (N0) or to distant sites (M0). |
| | M0 | |
| IB | T2 | The cancer is confined to the pancreas and is larger than 2 cm (0.8 inch) but no more |
| | N0 | than 4 cm (1.6 inches) across (T2). |
| | M0 | It has not spread to nearby lymph nodes (N0) or to distant sites (M0). |
| IIA | T3 | The cancer is confined to the pancreas and is bigger than 4 cm (1.6 inches) across (T3). |
| | N0 | It has not spread to nearby lymph nodes (N0) or to distant sites (M0). |
| | M0 | |
| IIB | T1 | The cancer is confined to the pancreas and is no bigger than 2 cm (0.8 inch) across |
| | N1 | (T1) AND it has spread to no more than 3 nearby lymph nodes (N1). |
| | M0 | It has not spread to distant sites (M0). |
| | T2 | The cancer is confined to the pancreas and is larger than 2 cm (0.8 inch) but no more |
| | N1 | than 4 cm (1.6 inches) across (T2) AND it has spread to no more than 3 nearby lymph |
| | M0 | nodes (N1). |
| | | It has not spread to distant sites (M0). |
| | T3 | The cancer is confined to the pancreas and is bigger than 4 cm (1.6 inches) across |
| | N1 | (T3) AND it has spread to no more than 3 nearby lymph nodes (N1). |
| | M0 | It has not spread to distant sites (M0). |
| III | T1 | The cancer is confined to the pancreas and is no bigger than 2 cm (0.8 inch) across |
| | N2 | (T1) AND it has spread to 4 or more nearby lymph nodes (N2). |
| | M0 | It has not spread to distant sites (M0). |
| | OR | |
| | T2 | The cancer is confined to the pancreas and is larger than 2 cm (0.8 inch) but no more |
| | N2 | than 4 cm (1.6 inches) across (T2) AND it has spread to 4 or more nearby lymph nodes |
| | M0 | (N2). |
| | | It has not spread to distant sites (M0). |
| | OR | |
| | T3 | The cancer is confined to the pancreas and is bigger than 4 cm (1.6 inches) across |
| | N2 | (T3) AND it has spread to 4 or more nearby lymph nodes (N2). |
| | M0 | It has not spread to distant sites (M0). |
| | OR | |
| | T4 | The cancer is growing outside the pancreas and into nearby major blood vessels (T4). |
| | Any N | The cancer may or may not have spread to nearby lymph nodes (Any N). |
| | M0 | It has not spread to distant sites (M0). |
| IV | Any T | The cancer has spread to distant sites such as the liver, peritoneum (the lining of the |
| | Any N | abdominal cavity), lungs or bones (M1). It can be any size (Any T) and might or might |
| | M1 | not have spread to nearby lymph nodes (Any N). |

According to a specific embodiment, the PDAC comprises intraductal papillary mucinous neoplasm.

In some embodiments, the pancreatic cancer is of an early stage, e.g., stage 1 (e.g., see 5 Table A IA, IB). In some embodiments, the pancreatic cancer is recurrent pancreatic cancer.

In some embodiments, the pancreatic cancer has reoccurred after remission.

In some embodiments, the individual has one or more metastatic tumors measurable, for example, by CT scan (or MRI).

In some embodiments, the pancreatic cancer is metastatic.

In some embodiments, the pancreatic cancer is unresectable pancreatic cancer. In some embodiments, the pancreatic cancer is a resectable pancreatic cancer.

In some embodiments, the pancreatic cancer is borderline resectable.

In some embodiments, the pancreatic cancer is characterized by germ-line mutations.

In some embodiments, the pancreatic cancer is characterized by lack of germ-line mutations.

According to a specific embodiment, the cancer is KRAS+.

In some embodiments, the primary location of the pancreatic cancer is the head of the pancreas.

As used herein “subject” refers to a mammal (e.g. human, dog, cat, horse, cow, sheep, pig or goat). According to a particular embodiment, the subject is a human.

According to some embodiments, the subject exhibits symptoms of pancreatic cancer.

According to some embodiments, the subject does not exhibit symptoms of pancreatic cancer.

According to a specific embodiment, the subject is diagnosed with pancreatic cancer.

According to a specific embodiment, the subject is not immune-deficient.

According to a specific embodiment, the subject does not have an active infectious disease, autoimmune or malignant disease other than pancreatic cancer

In some embodiments, the subject is a female.

In some embodiments, the subject is a male.

In some embodiments, the subject is under about 65 years old (such as under about any of 60, 55, 50, 45, or 40 years old).

In some embodiments, the subject is at least about 65 years old (such as at least about any of 70, 75, or 80 years old).

According to a specific embodiment, the subject is at least 18 years.

According to a specific embodiment, the subject has not been treated with any previous anti-cancer therapy, systemic or localized such as chemotherapy, radiotherapy, biological therapy, surgery and the like or for the pancreatic adenocarcinoma. Accordingly, treatment is first-line treatment.

In another embodiment, the subject is non-hospitalized.

For any of the aspects disclosed herein, the term “determining”, “measuring” or “measurement,” or alternatively “detecting” or “detection,” means assessing the presence, absence, quantity or amount (which can be an effective amount) of the determinant (e.g., SDC1) within a clinical or subject-derived sample, including the derivation of qualitative or quantitative concentration levels of such determinants.

As used herein “a blood sample or a fraction thereof” refers to a biological sample isolated from a subject and can include, by way of example and not limitation, whole blood or its fractions such as serum or plasma.

The sample may be a venous sample, peripheral blood mononuclear cell sample or a peripheral blood sample. In one embodiment, the sample comprises white blood cells including for example granulocytes, lymphocytes and/or monocytes. In one embodiment, the sample is depleted of red blood cells.

According to a specific embodiment, the sample is a serum sample.

As used herein “syndecan 1 (SDC1)” refers to a protein which in humans is\] encoded by the SDC1 gene. For other species, its orthologs are contemplated. According to a specific embodiment, the SDC1 is soluble SDC1.

As used herein “soluble SDC1” or “sSDC1” refers to levels (e.g., serum levels) of shedded SDC1-ectodomain (sSDC1). According to a specific embodiment, the maximal length of the ectodomain of SDC1 is 227-236 amino acids long.

TABLE B*

| | |
|---|---|
| Species | Human |
| ENTREZ | 6382 |
| Ensembl | ENSG00000115884 |
| UniProt | P18827 |
| RefSeq (mRNA) | NM_001006946 |
| | NM_002997 |
| RefSeq (protein) | NP_001006947 |
| | NP_002988 |

*content of accession numbers indicated is as of the date of filing of the international application).

As used herein “MMP7” also known as matrix metalloproteinase-7 (MMP-7), pump-1 protease (PUMP-1), or uterine metalloproteinase refers to an enzyme that in humans is encoded by the MMP7 gene. The enzyme (EC 3.4.24.23) has also been known as matrilysine, putative (or punctuated) metalloproteinase-1, matrix metalloproteinase pump 1, PUMP-1 proteinase, PUMP, metalloproteinase pump-1, putative metalloproteinase, MMP). Human MMP-7 has a molecular weight around 30 kDa.

Methods of measuring the level of protein determinants are well known in the art and include, e.g., immunoassays based on antibodies to proteins, aptamers or molecular imprints.

Protein determinants can be detected in any suitable manner, but are typically detected by contacting a sample from the subject with an antibody, which binds the protein determinant and then detecting the presence or absence of a reaction product. The antibody may be monoclonal, polyclonal, chimeric, or a fragment of the foregoing, and the step of detecting the reaction product may be carried out with any suitable immunoassay.

In one embodiment, the antibody which specifically binds the determinant is attached (either directly or indirectly) to a signal producing label, including but not limited to a radioactive label, an enzymatic label, a hapten, a reporter dye or a fluorescent label.

Immunoassays carried out in accordance with some embodiments of the present invention may be homogeneous assays or heterogeneous assays. In a homogeneous assay the immunological reaction usually involves the specific antibody (e.g., anti-determinant antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate, pipette tip or slide, and contacted with the specimen suspected of containing the antigen in a liquid phase.

The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

According to a specific embodiment, the method of determining the protein determinant(s) (e.g., SDC1) level is ELISA.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al., titled “Methods for Modulating Ligand-Receptor Interactions and their Application,” U.S. Pat. No. 4,659,678 to Forrest et al., titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” The determinant can also be detected with antibodies using flow cytometry. Those skilled in the art will be familiar with flow cytometric techniques which may be useful in carrying out the methods disclosed herein (Shapiro 2005). These include, without limitation, Cytokine Bead Array (Becton Dickinson) and Luminex technology.

Antibodies can be conjugated to a solid support suitable for a diagnostic assay (e.g., beads such as magnetic beads, protein A or protein G agarose, microspheres, plates, slides, pipette tip or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

In particular embodiments, the antibodies of the present invention are monoclonal antibodies.

Suitable sources for antibodies for the detection of determinants include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptide determinants described herein.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Those skilled in the art will be familiar with numerous suitable detectors that widely available from a variety of commercial sources and may be useful for carrying out the method disclosed herein. Commonly, an optical image of a substrate comprising bound labeling moieties is digitized for subsequent computer analysis. See generally The Immunoassay Handbook [The Immunoassay Handbook. Third Edition. 2005].

Examples of antibodies for measuring SDC1 include but are not limited to antibodies which bind the ectodomain of SDC1. Examples include, but are not limited to, Catalog No. ABIN6964046. Catalog No. ABIN1724936. ABIN1998767. Catalog No. ABIN2851944. ABIN1870672, ABIN1882216 Catalog No. ABIN3021805, ABIN2001267. ABIN5518953, all available from Antibodies on-line.

As mentioned, the determination of diagnosis, prognosis, treatment efficacy is done using SDC1 alone or in combination with other markers.

Preferably the combinations which are tested do not exceed 50, 40, 30, 20, 10, 9, 8, 7, 6, 5, 4, 3, or 2 markers. In another embodiment, no more than 20 protein markers are analyzed. In another embodiment, no more than 10 protein markers are analyzed. In another embodiment, no more than 9 protein markers are analyzed. In another embodiment, no more than 8 protein markers are analyzed. In another embodiment, no more than 7 protein markers are analyzed. In another embodiment, no more than 6 protein markers are analyzed in a single test/analysis, for the classification. In another embodiment, no more than 5 protein markers are analyzed. In another embodiment, no more than 4 protein markers are analyzed. In another embodiment, no more than 3 protein markers are analyzed. In another embodiment, no more than 2 protein markers are analyzed.

Examples of antibodies for measuring MMP7 are well known in the art such as for ELISA measurement available from R&D Systems.

As mentioned when a level of a determinant (e.g., SDC1) is above a predetermined threshold, the subject is diagnosed with pancreatic cancer.

As used herein “predetermined level” is with respect to the level of the determinant in the same sample (e.g., serum) of a control sample such as of subject(s) who do not have cancer, e.g., pancreatic cancer or of the same patient before treatment (such as when determining treatment efficacy.

As used herein “above” refers to at least 20%, 30%, 40%, 50%, 60%, 70%, 80%, 90%, 1.5 fold, 2 fold, 3 fold, 5 fold 7 fold 10 fold increase in SDC1 levels (other measured markers) with respect to the control.

Classification of subjects into subgroups according to the present invention is preferably done with an acceptable level of clinical or diagnostic accuracy. An “acceptable degree of diagnostic accuracy”, is herein defined as a test or assay (such as the test used in some aspects of the invention) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods determine severity with at least 75% total accuracy, more preferably 80%, 85%, 90%, 95%, 97%, 98%, 99% or greater total accuracy.

Alternatively, the methods determine severity with an MCC larger than 0.2, 0.3, 0.4, 0.5, 0.6, 0.7, 0.8, 0.9 or 1.0.

As used herein the term “diagnosing” refers to determining presence or absence of a pathology (e.g., a disease, disorder, condition or syndrome), classifying a pathology or a symptom, monitoring pathology progression, forecasting an outcome of a pathology and/or prospects of recovery and screening of a subject for a specific disease.

The term “prognosing survival” refers to assignment of the severity of the disease which may in one embodiment, relate to the probability to survive the cancer for a given time period e.g., 5 years.

A cutoff level of 30 ng/ml of serum soluble SDC-1 showed sensitivity of 75% and specificity of 75% in the derivation group, and sensitivity and specificity of 95% and 70%, respectively, in the validation group. A cutoff level of 26 ng/ml of serum SDC-1 showed sensitivity of 90% and specificity of 55% in the derivation group, and sensitivity and specificity of 100% and 55%, respectively, in the validation group.

Hence, according to some embodiments of the invention, the cutoff is 35 ng/ml.

According to another embodiment, treatment is first-line treatment against the pancreatic adenocarcinoma.

According to some embodiments of the invention, screening of the subject for a specific disease is followed by substantiation of the screen results using gold standard methods (imaging and/or molecular markers e.g., in situ, e.g., HPA, MMP and CA19-9)

The method of predicting the survival prognosis of a subject according to some embodiments of the invention enables the classification of a subject.

According to some embodiments of the invention, the method further comprising informing the subject of the diagnosis and/or the predicted prognosis of the subject.

As used herein the phrase “informing the subject” refers to advising the subject that based on the test result the subject should seek a suitable treatment regimen. Once the diagnosis/prognosis is determined, the results can be recorded in the subject's medical file, which may assist in selecting a treatment regimen and/or determining prognosis of the subject.

According to some embodiments of the invention, the method further comprising recording the diagnosis/prognosis/response to treatment of the subject in the subject's medical file.

As mentioned, the prediction of the diagnosis of a subject can be used to select the treatment regimen of a subject and thereby treat the subject in need thereof.

As used herein, the term “treating” includes abrogating, substantially inhibiting, slowing or reversing the progression of a condition, substantially ameliorating clinical symptoms of the metastatic pancreatic adenocarcinoma.

Examples of treatments for pancreatic cancer include, but are not limited to, surgery (palliative or potentially curative), ablation or embolization treatments, radiation therapy, chemotherapy, targeted therapy (e.g., EGFRi, PARPi), immunotherapy (e.g., anti PD1 or any other checkpoint inhibitors and the like).

It will be appreciated that the present teachings can also be used towards measuring treatment efficacy.

Thus, according to an aspect, there is provided a method of monitoring treatment of pancreatic cancer in a subject in need thereof, the method comprising:
(a) treating the subject with anti pancreatic cancer therapy (such as described above); and
(b) determining a level of syndecan 1 (SDC1) in a blood sample or a fraction thereof of the subject prior to and following (a), wherein when a decrease in SDC1 is determined an efficacious treatment is indicated.

As used herein “decrease” refers to at least 20%, 30%, 40%, 50%, 60%, 70%, 80%, 90%, 1.5 fold, 2 fold, 3 fold, 5 fold 7 fold 10 fold decrease in SDC1 levels with respect to the control (in this case, e.g., sample of the same subject which is otherwise the same as the test sample only it is taken prior to treating).

If no such decrease is indicated the physician should consider changing the treatment.

Some aspects of the invention also include a determinant-detection reagent such as antibodies packaged together in the form of a kit. The kit may contain in separate containers antibodies (e.g., bound to a solid matrix or packaged separately such as with reagents for binding them to the matrix), control formulations (positive and/or negative), and/or a detectable label such as fluorescein, green fluorescent protein, rhodamine, cyanine dyes, Alexa dyes, luciferase, radiolabels, among others. The detectable label may be attached to a secondary antibody which binds to the Fc portion of the antibody which recognizes the determinant. Instructions (e.g., written, tape, VCR, CD-ROM, etc.) for carrying out the assay may be included in the kit.

The kits of this aspect of the present invention may comprise additional components that aid in the detection of the determinants such as enzymes, salts, buffers etc. necessary to carry out the detection reactions.

For example, determinant detection reagents (e.g. antibodies) can be immobilized on a solid support such as a porous strip or an array to form at least one determinant detection site. The measurement or detection region of the porous strip may include a plurality of sites. A test strip may also contain sites for negative and/or positive controls. Alternatively, control sites can be located on a separate strip from the test strip. Optionally, the different detection sites may contain different amounts of immobilized detection reagents, e.g., a higher amount in the first detection site and lesser amounts in subsequent sites. Upon the addition of test sample, the number of sites displaying a detectable signal provides a quantitative indication of the amount of determinants present in the sample. The detection sites may be configured in any suitably detectable shape and are typically in the shape of a bar or dot spanning the width of a test strip.

Polyclonal antibodies for measuring determinants include without limitation antibodies that were produced from sera by active immunization of one or more of the following: Rabbit, Goat, Sheep, Chicken, Duck, Guinea Pig, Mouse, Donkey, Camel, Rat and Horse.

Examples of detection agents, include without limitation: scFv, dsFv, Fab, sVH, F(ab′)₂, Cyclic peptides, Haptamers, A single-domain antibody, Fab fragments, Single-chain variable fragments, Affibody molecules, Affilins, Nanofitins, Anticalins, Avimers, DARPins, Kunitz domains, Fynomers and Monobody.

In particular embodiments, the kit does not comprise a number of antibodies that specifically recognize more than 50, 20 15, 10, 9, 8, 7, 6, 5 or 4 polypeptides.

In other embodiments, the array of the present invention does not comprise a number of antibodies that specifically recognize more than 50, 20 15, 10, 9, 8, 7, 6, 5 or 4 polypeptides.

The kit may comprise a control blood or a blood fraction sample of a subject diagnosed with pancreatic cancer that may be combined with at least an antibody to SDC1 (and optionally to other protein determinants e.g., MMP7).

A machine-readable storage medium can comprise a data storage material encoded with machine-readable data or data arrays which, when using a machine programmed with instructions for using the data, is capable of use for a variety of purposes. Measurements of effective amounts of the biomarkers of the invention and/or the resulting evaluation of risk from those biomarkers can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system used in some aspects of the invention may also be considered to be implemented as a computer-readable storage medium, configured with a computer program, where the storage medium so configured causes a computer to operate in a specific and predefined manner to perform various functions described herein.

The polypeptide determinants of the present invention, in some embodiments thereof, can be used to generate a “reference determinant profile” of those subjects who do not have cancer, e.g., pancreatic cancer. The determinants disclosed herein can also be used to generate a “subject determinant profile” taken from subjects who have cancer, e.g., pancreatic cancer. The subject determinant profiles can be compared to a reference determinant profile to diagnose or identify subjects with pancreatic cancer. The reference and subject determinant profiles of the present invention, in some embodiments thereof, can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Methods

### Design and Patient Population (for Examples 1-5)

This was a case-control study conducted at Sheba Medical Center, a tertiary academic center in Israel. Patients with a new diagnosis of EUS-guided or surgically obtained histologically proven PDAC were included in the study. The derivation cohort comprised of newly-diagnosed PDAC patients and healthy control individuals prospectively and sequentially recruited between October 2019 and October 2020. A validation cohort was thereafter enrolled comprising additional prospectively recruited healthy individuals as well as PDAC patients' serum samples obtained from the Sheba Medical Centers Tissue bank repository. For the purpose of subgroup assessment vis-à-vis survival outcomes, these two cohorts were unified into one group and enriched with additional PDAC patients who underwent upfront surgery between September 2014-October 2019 whose serum were similarly stored in the Sheba Tissue bank.

In all patients, serum tests for the tumor markers CA 19-9 and SDC1 analysis were performed at baseline before any surgical or oncological treatment. Retrieved data from medical files included demographics, smoking habits, presence of diabetes, germline testing (if performed), localization of tumor, clinical/pathological staging, performance of surgery and censor date (December 2020)/death. Patients were excluded from the study if they were unable to provide informed consent or suffered from systemic active infectious disease, autoimmune disorder or other known extra-pancreatic malignancy. All patients signed an informed consent (either for this study or for tissue bank), and the study was approved by the institutional ethics review board.

### Staging of Tumor

Staging of PDAC was classified either by TMN system/American Joint Committee on Cancer (AJCC) Staging for PDAC, or by three clinically distinct patient groups-resectable (T1-3, stages 1 and 2), locally advanced (T4, stage 3) and metastatic disease (M1, stage 4). Staging was determined on the basis of imaging modalities (typically CT scans) for non-resectable tumors, and pathologically-determined for resectable ones. For locally advanced tumors in which neo-adjuvant treatment was given before surgery, we considered pre-operative staging for the purpose of categorization.

### Study Endpoints

The primary outcome was the diagnostic accuracy of serum SDC1 to differentiate between patients with PDAC and healthy individuals. Secondary outcomes included the utility of this biomarker to distinguish between different tumor stages and its association with survival outcomes and with patient and tumor characteristics.

### Soluble Syndecan-1 Analysis

Venous blood was collected, centrifuged at 3000×g for 10 min, and obtained serum then stored at −80° C. Samples provided by Sheba Pancreatic Cancer biorepository were stored in 80 C at all times, following serum extraction. Serum SDC1 concentrations were determined using human syndecan-1 enzyme-linked immunosorbent assay (ELISA; Diaclone Research, Besancon, France) according to the manufacturer's instructions. Samples, standards and diluted biotinylated antibody were added to pre-coated wells (in duplicates) and incubated for 1 hour at room temperature. The buffer that was used for washes supplied with the Diaclone s-CD138 kit. The wash buffer was diluted 200-fold in distilled water in the same day of the assay. After 3 washes, horseradish-peroxidase-streptavidin conjugate was added, and the plates were incubated for 30 minutes at room temperature. Substrate was added and the color was allowed to develop for 10-15 minutes. The reaction was stopped with sulfuric acid and the absorbance was read at 450 nm. On microtiter plate ELISA reader. TECAN, Neotec, fitted with appropriate filters-450 nm with 620 nm reference filter. SSDC1 concentrations were reported as ng/ml and determined blindly without knowledge of the clinical data.

### Statistical Analysis

Categorical variables were described as frequency and percentage and compared using Chi-square test or Fisher's exact test. Continuous variables were described as median and interquartile range (IQR) and compared between categories using Mann-Whitney U test and Kruskal-Wallis test. Associations between SDC-1 levels and continuous variables were assessed using Spearman's correlation Coefficient and associations between SDC-1 levels and categorical variables were assessed using Mann-Whitney U test. Receiver operating characteristic (ROC) analysis and the Youden index were used to find an optimal cut-off value. Survival during the follow-up period was analyzed by Kaplan-Meier curve, and log rank test was used to compare between categories. Length of follow-up was evaluated using reverse censoring method. Sample size was calculated using area under the curve 0.85, ratio of PDAC patients to controls 2:1 and 95% confidence interval with a width of 0.2. According to these assumptions 37 PDAC patients and 19 controls were needed. All statistical tests were two sided. p<0.05 was considered as statistically significant. All statistical analysis was performed using SPSS (IBM SPSS Statistics for Windows, version 25, IBM Corp, Armonk, NY, USA). Area under the curve (AUC) was evaluated using survival-ROC package version 1.0.3 in R: a language and environment for statistical computing (The R foundation for statistical computing version 3.3.3, 2017).

### Example 2

### Patient Characteristics

The derivation cohort comprised 39 patients and 20 healthy individuals and the validation cohort included 38 patients and 20 healthy individuals. Patient characteristics in the derivation and validation groups are shown in Table 1 below.

TABLE 1
Patient demographics and baseline characteristics
(for which results are shown).

| | Derivation cohort | | Validation cohort | | |
|---|---|---|---|---|---|
| | PDAC | Control | PDAC | Control | P |
| | N = 39 | N = 20 | N = 38 | N = 20 | value ^{#} |
| Age, median | 68 | 31 | 70 | 26 | 0.617 |
| (IQR), years | (63-74) | (27-36)* | (64-75) | (21-35)* | |
| Male gender, | 19 (48.7) | 9 (45.0) | 22 (57.9) | 10 (50.0) | 0.420 |
| N (%) | | | | | |
| Diabetes, N (%) | 22 (56.4) | | 18 (47.4) | | 0.427 |
| Smoking, N (%) | 13 (33.3) | | 12 (31.6) | | 0.869 |
| Tumor | | | | | |
| localization, N (%) | | | | | |
| Head | 30 (76.9) | | 25 (65.8) | | 0.06 |
| Body/tail | 9 (23.1) | | 13 (34.2) | | |
| Staging, N (%) | | | | | |
| 1 | 4 (10.3) | | 6 (15.8) | | 0.376 |
| 2 | 11 (28.2) | | 12 (31.6) | | 0.764 |
| 3 | 4 (10.3) | | 5 (13.2) | | 0.576 |
| 4 | 20 (51.3) | | 15 (39.5) | | 0.298 |
| Patient group | | | | | |
| Resectable | | | | | |
| Locally | | | | | |
| advanced | | | | | |
| Metastatic | | | | | |
| Germline | 3 (7.7) | | 5 (13.2) | | 0.970 |
| mutation, N (%) | | | | | |
| CA 19-9 ≥37 | 31 (79.5) | | 33 (86.8) | | 0.389 |
| U/mL, N (%) | | | | | |
| Baseline CA 19-9, | 392 (71- | | 394 (92- | | 0.854 |
| median (IQR), | 3198) | | 1502) | | |
| U/mL | | | | | |
| Surgery, N (%) | 9 | | 20 | | 0.007 |
| Upfront | 5 | | 20 | | |
| After neo-adj. | 4 | | 0 | | |

*P value <0.0001 between PDAC and control patients
^{#} Between derivation and validation cohorts
PDAC—pancreatic ductal adenocarcinoma; IQR—interquartile range 25-75

The median age of patients with PDAC was 68 and 70 years in these groups, respectively. Patients with PDAC were significantly older than healthy controls in both groups. Patient characteristics, tumor location, tumor staging and level of CA 19-9 were comparable between derivation and validation groups. Actionable germline pathogenic variants were found in 8 patients in the two groups (BRCA 1/2—6, MSH6—1, CHEK2—1). Significantly more patients with PDAC in the validation group underwent surgery compared with the derivation group (20 vs. 9, P=0.007), including 3, 5 and 1 patients in stage-disease I, II and III, respectively, in the derivation group, and 6, 11 and 3 patients, respectively, in the validation group.

### Example 3

### Serum Syndecan-1 Level Diagnostic Accuracy for PDAC-Primary Outcome

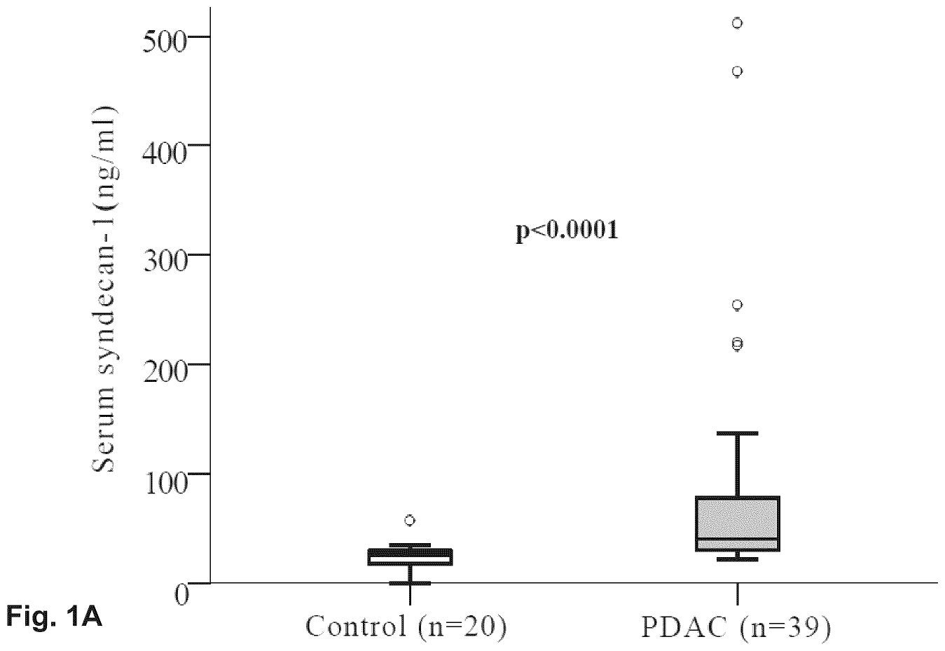

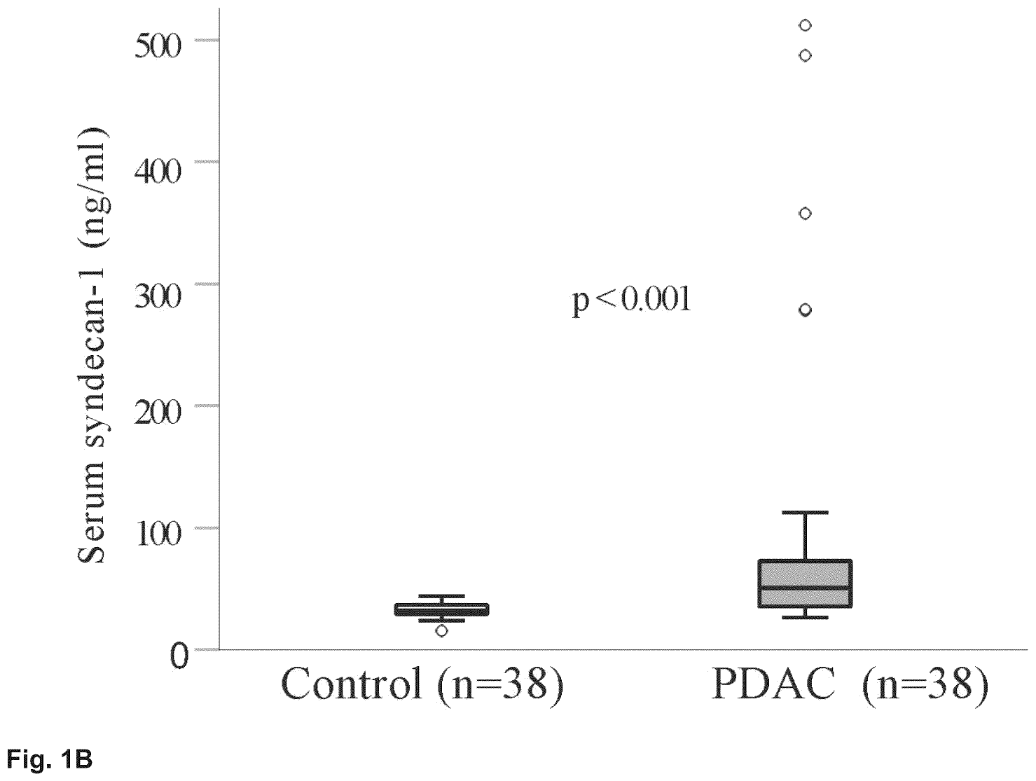

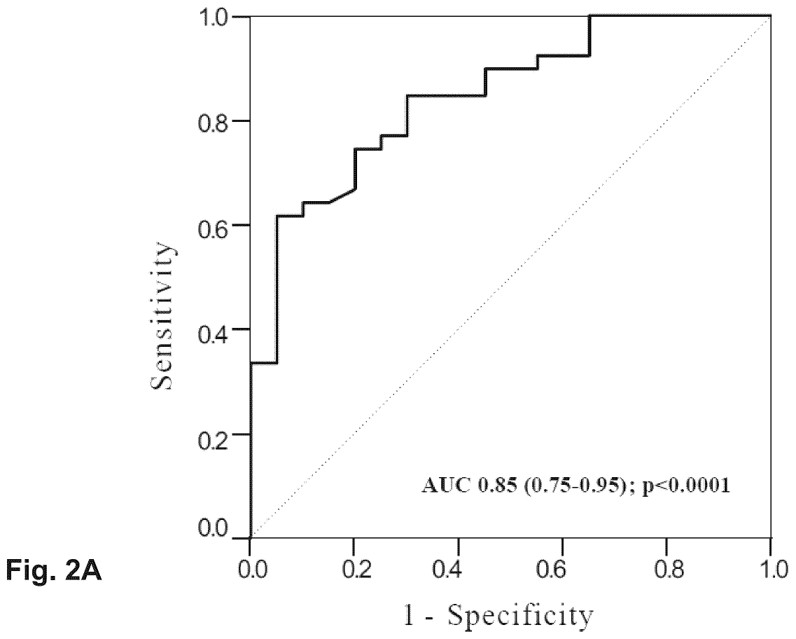

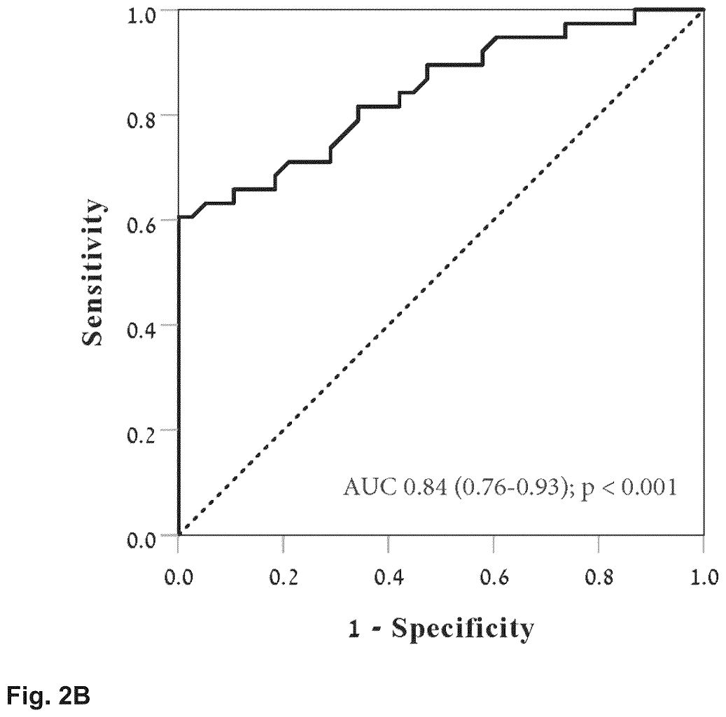

In the deviation cohort, median serum SDC-1 level was significantly higher in the PDAC group compared with healthy controls (40.1 ng/ml, IQR 29.8-95.3 vs. 25.6 ng/ml, IQR 17.1-29.8, respectively; p<0.0001, FIG. 1A). Upon ROC analysis, the area under the curve (AUC) was 0.847 (95% confidence interval (CI) 0.747 to 0.947, p value <0.0001, FIG. 2A).

In the validation cohort median serum SDC-1 level was also significantly higher in the PDAC group compared with healthy controls (50.5 ng/ml, IQR 35.1-73.2 vs. 25.2 ng/ml, IQR 22.8-31.7, respectively; p<0.0001) (FIG. 1B). The ROC analysis AUC was 0.904 (95% CI 0.818 to 0.987, p value <0.0001) in the validation cohort (FIG. 2B).

A cutoff level of 30 ng/ml of serum SDC-1 showed sensitivity of 75% and specificity of 75% in the derivation group, and sensitivity and specificity of 95% and 70%, respectively, in the validation group. A cutoff level of 26 ng/ml of serum SDC-1 showed sensitivity of 90% and specificity of 55% in the derivation group, and sensitivity and specificity of 100% and 55%, respectively, in the validation group.

### Example 4

### Diagnostic Utility of Serum Syndecan-1 for PDAC Staging—Secondary Outcome

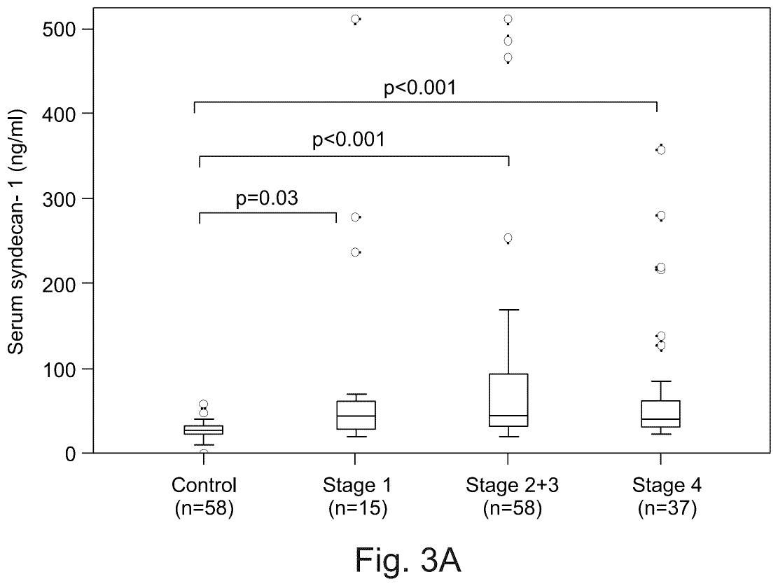

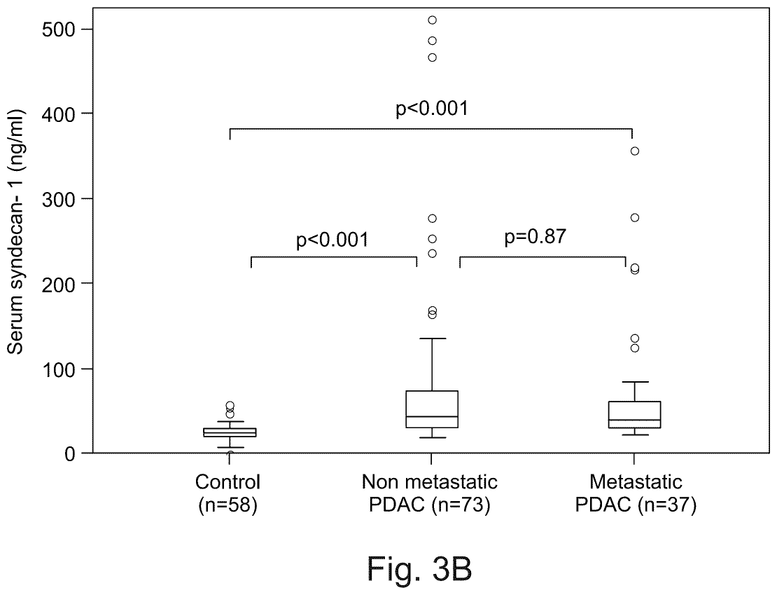

In order to evaluate the diagnostic value of SDC-1 for PDAC staging, the cohort was enriched with early-stage patients undergoing upfront surgery (n=33), generating a combined total cohort size of 110 patients. Serum SDC-1 level was not significantly different among patients with different tumor stages [median, (IQR): stage 1 (N=15)-43.7 ng/ml (24.8-68.6); stage 2 (N=44)-45.5 ng/ml (30.2-95.0); stage 3 (N=14)-37.4 ng/ml (32.1-59.7); stage 4 (N=37)-40.2 ng/ml (31.2-63.7); P=0.854] (FIG. 3A). Likewise, SDC1 level was not significantly different when comparing metastatic (n=37) vs. non-metastatic (n=73) PDAC groups [median (IQR) 40.2 ng/ml (31.2-63.7) vs. median (IQR) 43.7 ng/ml (31.3-84.0), respectively; P=0.877) (FIG. 3B).

### Example 5

### Serum Syndecan-1 for Association Analysis and Prediction of Survival

Association between baseline characteristics, tumor location, CA 19-9 level and serum SDC-1 are presented in Table 2. Serum SDC-1 was higher in patients with tumor location in the head of pancreas vs. body/tail localization (P=0.019), and was not significantly associated with age, gender, smoking habits, diabetes, germline mutation and elevated CA 19-9 level (≥37 U/mL). However, Stage IV disease was equally distributed between patients with pancreatic head or body/tail localization (71.8% vs. 53.1%, respectively, P value=0.076). Remarkably, a subset of patients with normal serum CA19-9 (<37 U/mL) had elevated serum level of SDC-1; Twenty out of 110 patients (18.1%) had normal serum CA19-9, of whom 5 (25%) had metastatic disease and the rest (75%) had stage 1-2 disease. Twelve of these 20 patients (60%) had serum SDC-1≥35 ng/ml, of whom 3 (25%) had metastatic disease and the rest had stage 1-2 disease.

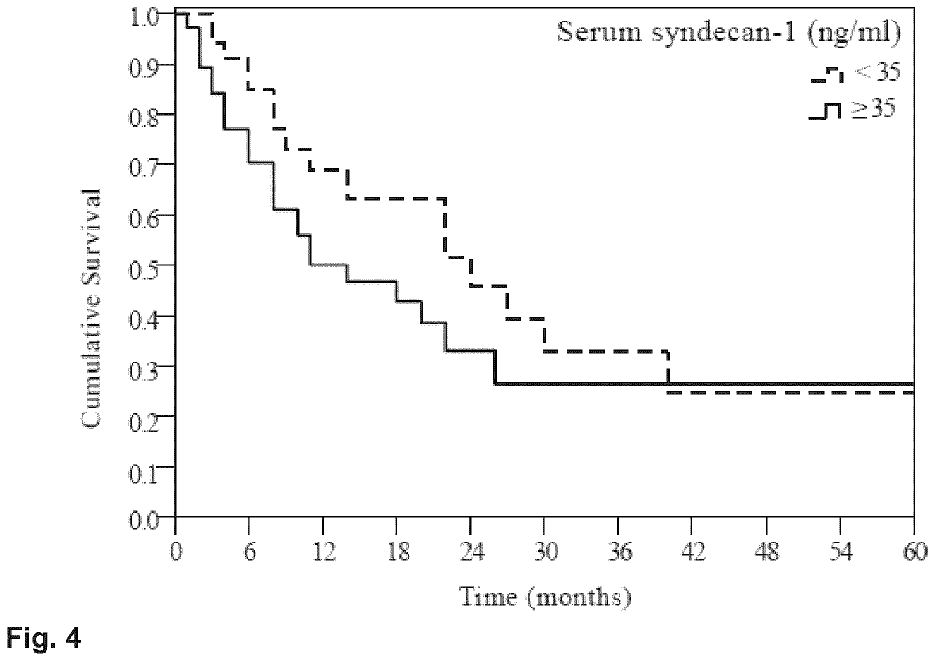

Overall, 47/110 (42.7%) of patients survived, with a median time of follow-up of 11 months (IQR 5-28). Kaplan-Meyer analysis of overall survival during the follow-up period (FIG. 4), showed that the median overall survival of patients with baseline serum SDC-1 <35 ng/ml vs. ≥35 ng/ml was not significantly different [24.0 months (CI-12.1-35.8) vs. 14.0 months (CI 4.7-23.2), respectively; P=0.153], although there was a trend towards better survival after 12 months in patients with serum levels <35 ng/ml (P=0.066, a period which is meaningful in such an aggressive disease.

TABLE 2

| | |
|---|---|
| Serum syndecan-1 ng/ml, | |
| Median (IQR) | P value |

| | | |
|---|---|---|
| Age | 0.003 * | 0.975 |

| | | | |
|---|---|---|---|
| Gender | | | 0.792 |
| Male | 43.7 | (32.2-69.6) | |
| Female | 40.2 | (29.0-73.3) | |
| Smoking | | | 0.556 |
| Yes | 41.8 | (30.6-63.0) | |
| No | 43.7 | (31.2-80.2) | |
| Diabetes | | | 0.744 |
| Yes | 42.3 | (31.0-87.1) | |
| No | 43.5 | (32.1-67.9) | |
| Germline mutation | | | 0.818 |
| Yes | 45.2 | (32.3-110.7) | |
| No | 43.0 | (30.3-71.7) | |
| Localization of PDAC | | | 0.019 |
| Head | 48.0 | (32.4-94.6) | |
| Body/tail | 37.2 | (28.9-49.3) | |

| | | |
|---|---|---|
| Tumor size | −0.006 * | 0.946 |

| | | | |
|---|---|---|---|
| CA 19-9 ≥ 37 U/mL | | | 0.190 |
| Yes | 43.7 | (32.3-75.7) | |
| No | 37.2 | (28.9-52.5) | |

* Data are presented as Spearman's rank correlation coefficient
PDAC— pancreatic ductal adenocarcinoma;
IQR—interquartile range 25-75

### Example 6

The study presented hereinabove in Examples 1-5 was repeated with an age-matched validation cohort (see Table 3 below). Similar results were obtained.

TABLE 3
Patient demographics and baseline characteristics

| | Derivation cohort | | | | Validation cohort | | | | |
|---|---|---|---|---|---|---|---|---|---|
| | PDAC | | Control | | PDAC | | Control | | |
| | N = 39 | | N = 20 | | N = 38 | | N = 38 | | P value ^{#} |
| Age, median (IQR), | 68 | (63-74) | 31 | (27-36) * | 70 | (64-75) | 70 | (65- 75) ** | 0.617 |
| years | | | | | | | | | |
| Male gender, N (%) | 19 | (48.7) | 9 | (45.0) ** | 22 | (57.9) | 17 | (44.7) ** | 0.420 |
| Diabetes, N (%) | 22 | (56.4) | | | 18 | (47.4) | 13 | (34.2) ** | 0.427 |
| Smoking, N (%) | 13 | (33.3) | | | 12 | (31.6) | 4 | (11.0) \hat{ } | 0.869 |
| Tumor localization, N (%) | | | | | | | | | |
| Head | 30 | (76.9) | | | 25 | (65.8) | | | 0.06 |
| Body/tail | 9 | (23.1) | | | 13 | (34.2) | | | |
| Staging, N (%) | | | | | | | | | |
| 1 | 4 | (10.3) | | | 6 | (15.8) | | | 0.376 |
| 2 | 11 | (28.2) | | | 12 | (31.6) | | | 0.764 |
| 3 | 4 | (10.3) | | | 5 | (13.2) | | | 0.576 |
| 4 | 20 | (51.3) | | | 15 | (39.5) | | | 0.298 |
| Tumor size. median | 28 | (23-40) | | | 30 | (25-41) | | | 0.865 |
| (IQR), mm | | | | | | | | | |
| Germline mutation, N | 3 | (7.7) | | | 5 | (13.2) | | | 0.970 |
| (%) | | | | | | | | | |
| CA 19-9 ≥ 37 U/mL, N | 31 | (79.5) | | | 33 | (86.8) | | | 0.389 |
| (%) | | | | | | | | | |
| Baseline CA 19-9, | 392 | (71-3198) | | | 394 | (92-1502) | | | 0.854 |
| median (IQR), U/mL | | | | | | | | | |

| | | | |
|---|---|---|---|
| Surgery, N (%) | 9 | 20 | 0.007 |
| Upfront | 5 | 20 | |
| After neo-adj. | 4 | 0 | |

* P value < 0.001 between PDAC and control patients
** P value - not significant between PDAC and control patients
\hat{ } P value = 0.03 between PDAC and control patients
^{#} between derivation and validation PDAC cohorts
PDAC—pancreatic ductal adenocarcinoma;
IQR—interquartile range 25-75

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-2. (canceled)

3. A method of treating pancreatic cancer in a subject in need thereof, the method comprising:
(a) diagnosing pancreatic cancer in the subject by determining a level of at least syndecan 1 (SDC1) in a blood sample or a fraction thereof of the subject, wherein when said level of SDC1 is above a predetermined threshold, the subject is diagnosed with pancreatic cancer; and
(b) treating the subject diagnosed with pancreatic cancer with an anti pancreatic cancer therapy or selecting a treatment with an anti pancreatic cancer therapy.

4. A method of monitoring treatment of pancreatic cancer in a subject in need thereof, the method comprising:
(a) treating the subject with anti pancreatic cancer therapy;
(b) determining a level of at least syndecan 1 (SDC1) in a blood sample or a fraction thereof of the subject prior to and following (a), wherein when a decrease in SDC1 is determined an efficacious treatment is indicated.

5. The method of claim 3, further comprising determining a level of MMP7, wherein when said level of SDC1 and said MMP7 are above a predetermined threshold, the subject is diagnosed with pancreatic cancer.

6. A composition of matter comprising a blood sample or a fraction thereof of a subject and an antibody to SDC1.

7. The composition of claim 6, wherein said blood sample of fraction thereof is of a subject having pancreatic cancer.

8. The method of claim 3, wherein said pancreatic cancer is pancreatic ductal adenocarcinoma (PDAC).

9. The method of claim 3, wherein said level is protein level.

10. The method of claim 9, wherein said determining is by ELISA.

11. The method of claim 3, wherein said pancreatic cancer is KRAS+.

12. The method of claim 3, wherein said pancreatic cancer is resectable.

13. The method of claim 3, wherein said treating is by a surgery.

14. The method of claim 3, wherein a location of said cancer is in the head of the pancreas.

15. The method of claim 3, further comprising corroboration of said pancreatic cancer or treatment thereof with imaging or molecular markers.

16. The method of claim 15, wherein said molecular markers are selected from the group consisting of HPA, MMP and CA 19-9.

17. The method of claim 3, wherein said subject does not suffer from an autoimmune disease, a systemic active infectious disease and/or an extra-pancreatic malignancy.
